(19) United States
(12) Patent Application Publication
(10) Pub. No.: US 2024/0131000 A1
(43) Pub. Date: Apr. 25, 2024

(54) AGENTS AND METHODS FOR PREVENTING AND TREATING SKELETAL MUSCLE DISEASES

(71) Applicant: The Regents Of the University of California, Oakland, CA (US)

(72) Inventor: Stephen C. CANNON, Los Angeles, CA (US)

(73) Assignee: The Regents Of the University of California, Oakland, CA (US)

(21) Appl. No.: 18/383,256

(22) Filed: Oct. 23, 2023

Related U.S. Application Data
(60) Provisional application No. 63/418,859, filed on Oct. 24, 2022.

(51) Int. Cl. A61K 31/27; A61K 45/06; A61P 21/00

(52) U.S. Cl. CPC A61K 31/27; A61K 45/06; A61P 21/00

(57) ABSTRACT

Methods are described for preventing or treating skeletal diseases such as period paralysis by administering to a subject a Kv7 channel opener.

### CROSS-REFERENCE TO RELATED APPLICATION

This application claims benefit of and priority to U.S. Provisional Patent Application Ser. No. 63/418,859, filed Oct. 24, 2022, which is incorporated herein by reference in its entirety.

### GOVERNMENT SUPPORT

This invention was made with government support under Grant Number AR078198, awarded by the National Institutes of Health. The government has certain rights in the invention.

### BACKGROUND

Periodic paralysis (PP) is a rare genetic disorder that causes sudden attacks of short-term muscle weakness, stiffness, or paralysis. These attacks may affect the whole body or just 1 or 2 limbs. There are several different forms of PP: they all involve defects in ion channels. PP attacks can start in childhood or adulthood. They may happen after hard exercise or other triggers. Depending on the form of PP, symptoms may be mild or severe, and they may last for minutes or days. Sometimes, the disease may slowly get worse over time and cause permanent muscle damage.

The main forms of PP are: (1) Hypokalemic PP (hypoPP). Attacks may result from low blood levels of potassium. Other triggers may include strenuous exercise, foods with a lot of sugars and starches (carbohydrates), licorice, stress, cold temperatures, and certain medicines. This is the most common form of familial PP. (2) Hyperkalemic PP (hyperPP). Attacks often occur in association with high blood levels of potassium. Other triggers may include strenuous exercise, fasting, stress, cold, and certain medicines. (3) Thyrotoxic PP. Attacks may result from high levels of thyroid hormone, usually endogenous thyrotoxicosis but exogenous administration may also cause PP. Triggers may include exercise, meals with a lot of carbohydrates, and stress. This form appears mostly in men, especially in those of Asian background. (4) Andersen-Tawil syndrome (ATS). Attacks may result from swings in potassium blood levels. Triggers may also include exercise, stress, and certain medicines. People with this form of PP also often have cardiac arrhythmias and a certain set of skeletal features. These include a broad forehead, widely spaced eyes, low-set ears, a small chin, curved fingers, webbed toes.

Hypokalemic periodic paralysis (HypoPP) presents with recurrent episodes weakness, often in association with low serum potassium ([K+]<3.5 mEq/1). Attacks of weakness are variable, both amongst affected members in a family or for a single individual over time, with regard to affected muscle groups, severity, frequency and duration. Trigger factors that increase the risk of experiencing an attack of weakness are a prominent feature in all forms of periodic paralysis, with the events in HypoPP being carbohydrate-rich meals, rest after exercise, or stress. Consequently, the first approach to disease management is life-style changes to minimize the occurrence of trigger events. When these measures are insufficient, then an escalating level of pharmacological interventions is used beginning with oral K supplements, then K-sparing diuretics, and finally carbonic anhydrase inhibitors. While benefit has been established in double-blind placebo-controlled trials, there is an important unmet need with about 50% of patients on carbonic anhydrase inhibitors receiving inadequate control of episodic weakness or unable to tolerate these medications.

Hyperkalemic periodic paralysis (HyperPP) is characterized by recurrent episodes of weakness that are often triggered by strenuous exercise, fasting, or stress. The first-line approach for symptom management is avoiding triggers or ingestion of a carbohydrate snack to promote lowering of serum potassium. These maneuvers may be insufficient for optimal quality of life, and so pharmacologic interventions are added. Potassium wasting diuretics (e.g., hydrochlorothiazide) are tried first, followed by carbonic anhydrase inhibitors if needed.

New methods for treating HypoPP among other forms of periodic paralysis, and for skeletal muscle diseases, are needed.

### SUMMARY

In one aspect, a method is provided for preventing or treating a skeletal muscle disease in a subject comprising administering to the subject an effective amount of a potassium channel opener. In certain embodiments, the skeletal muscle disease is an inherited skeletal muscle disease or a sporadic skeletal muscle disease. In certain embodiments, the inherited skeletal muscle disease is periodic paralysis. In certain embodiments, the periodic paralysis is hypokalemic periodic paralysis, hyperkalemic periodic paralysis, Andersen Tawil syndrome (ATS), or thyrotoxic periodic paralysis. In certain embodiments, the thyrotoxic periodic paralysis is endogenous or caused by exogenous thyroid hormone administration.

In certain embodiments, the potassium channel opener is a Kᵥ7 channel opener. In certain embodiments, the Kᵥ7 channel opener is a KCNQ2/3 (Kᵥ7.2/7.3), KCNQ2, KCNQ3, KCNQ4 or KCNQ5 potassium channel opener or any combination thereof. In certain embodiments, the Kᵥ7 channel opener is selected from retigabine, (10,10-bis(4-pyridinylmethyl)-9(10H)-anthracenone dihydrochloride), N-[4-(6-fluoro-3,4-dihydro-1H-isoquinolin-2-yl)-2,6-dimethylphenyl]-3,3-dimethylbutanamide, (N-[(3-fluorophenyl)-methyl]-1-(2-methoxyethyl)-4-methyl-2-oxo-(7-trifluoromethyl)-1H-quinoline-3-carboxylic acid amide), N-[4-(6-fluoro-3,4-dihydro-1H-isoquinolin-2-yl)-2,6-dimethylphenyl]-3,3-dimethylbutanamide or flupirtine, or any analogue of any of the foregoing.

In certain embodiments, the effective amount comprises administering the potassium channel opener from between about 30 minutes prior to consuming food until 12 hours after consuming food.

In certain embodiments, the preventing or treating is abortive therapy. In certain embodiments, the abortive therapy is achieved with a single dose of potassium channel opener. In certain embodiments, the potassium channel opener is retigabine, (10,10-bis(4-pyridinylmethyl)-9(10H)-anthracenone dihydrochloride), N-[4-(6-fluoro-3,4-dihydro-1H-isoquinolin-2-yl)-2,6-dimethylphenyl]-3,3-dimethylbutanamide, (N-[(3-fluorophenyl)-methyl]-1-(2-methoxyethyl)-4-methyl-2-oxo-(7-trifluoromethyl)-1H-quinoline-3-carboxylic acid amide), N-[4-(6-fluoro-3,4-dihydro-1H-isoquinolin-2-yl)-2,6-dimethylphenyl]-3,3-dimethylbutanamide or flupirtine, or any analogue of any of the foregoing.

In certain embodiments, the preventing or treating therapy is prophylactic therapy. In certain embodiments, the potassium channel opener is retigabine, (10,10-bis(4-pyridinylmethyl)-9(10H)-anthracenone dihydrochloride), N-[4-(6-fluoro-3,4-dihydro-1H-isoquinolin-2-yl)-2,6-dimethylphenyl]-3,3-dimethylbutanamide, (N-[(3-fluorophenyl)-methyl]-1-(2-methoxyethyl)-4-methyl-2-oxo-(7-trifluoromethyl)-1H-quinoline-3-carboxylic acid amide), N-[4-(6-fluoro-3,4-dihydro-1H-isoquinolin-2-yl)-2,6-dimethylphenyl]-3,3-dimethylbutanamide or flupirtine, or any analogue of any of the foregoing.

In certain embodiments, the method further comprises administering to the subject a potassium-sparing diuretic, a potassium supplement, a carbonic anhydrase inhibitor, a sodium channel blocker, or any combination thereof. In certain embodiments, the potassium-sparing diuretic is amiloride, eplerenone, spironolactone or triamterene, or any analogue thereof. In certain embodiments, the carbonic anhydrase inhibitor is selected from is acetazolamide, topiramate, methazolamide, zonisamide, sulthiame or dichlorphenamide, or an analogue of any of the foregoing. In certain embodiments, the method further comprises administering to the subject a sodium channel blocker. In certain embodiments, the sodium channel blocker is mexiletine or flecainide, or an analogue thereof. In certain embodiments, the periodic paralysis is hyperkalemic periodic paralysis.

In one aspect, a therapeutic combination is provided comprising a potassium channel opener and a carbonic anhydrase inhibitor. In certain embodiments, the potassium channel opener is retigabine, (10,10-bis(4-pyridinylmethyl)-9(10H)-anthracenone dihydrochloride), N-[4-(6-fluoro-3,4-dihydro-1H-isoquinolin-2-yl)-2,6-dimethylphenyl]-3,3-dimethylbutanamide, (N-[(3-fluorophenyl)-methyl]-1-(2-methoxyethyl)-4-methyl-2-oxo-(7-trifluoromethyl)-1H-quinoline-3-carboxylic acid amide), N-[4-(6-fluoro-3,4-dihydro-1H-isoquinolin-2-yl)-2,6-dimethylphenyl]-3,3-dimethylbutanamide or flupirtine, or any analogue of any of the foregoing. In certain embodiments, the carbonic anhydrase inhibitor is acetazolamide, topiramate, methazolamide, zonisamide, sulthiame, or dichlorphenamide, or an analogue of any of the foregoing. In certain embodiments, the therapeutic combination is a synergistic combination.

In certain embodiments, the therapeutic combination further comprises a potassium supplement, a potassium sparing diuretic, a sodium channel blocker, or any combination thereof. In certain embodiments, the potassium-sparing diuretic is amiloride, eplerenone, spironolactone or triamterene, or an analogue of any of the foregoing. In certain embodiments, the sodium channel blocker is mexiletine or flecainide, or an analogue of any of the foregoing.

In one aspect, a therapeutic combination is provided comprising a potassium channel opener and a potassium supplement. In certain embodiments, the potassium channel opener is retigabine, XE991, XEN1101, XEN496, GRT-X, N-[4-(6-fluoro-3,4-dihydro-1H-isoquinolin-2-yl)-2,6-dimethylphenyl]-3,3-dimethylbutanamide or flupirtine, or an analogue of any of the foregoing. In certain embodiments, the therapeutic combination is a synergistic combination. In certain embodiments, the therapeutic combination further comprises a carbonic anhydrase inhibitor, a potassium sparing diuretic, a sodium channel blocker, or any combination thereof. In certain embodiments, the carbonic anhydrase inhibitor is selected from is acetazolamide, topiramate, methazolamide, zonisamide, sulthiame or dichlorphenamide, or an analogue of any of the foregoing. In certain embodiments, the potassium-sparing diuretic is amiloride, eplerenone, spironolactone or triamterene, or an analogue of any of the foregoing. In certain embodiments, the sodium channel blocker is mexiletine or flecainide, or an analogue of any of the foregoing.

In one aspect, a therapeutic combination is provided comprising a potassium channel opener and a potassium sparing diuretic. In certain embodiments, the potassium channel opener is retigabine, (10,10-bis(4-pyridinylmethyl)-9(10H)-anthracenone dihydrochloride), N-[4-(6-fluoro-3,4-dihydro-1H-isoquinolin-2-yl)-2,6-dimethylphenyl]-3,3-dimethylbutanamide, (N-[(3-fluorophenyl)-methyl]-1-(2-methoxyethyl)-4-methyl-2-oxo-(7-trifluoromethyl)-1H-quinoline-3-carboxylic acid amide), N-[4-(6-fluoro-3,4-dihydro-1H-isoquinolin-2-yl)-2,6-dimethylphenyl]-3,3-dimethylbutanamide or flupirtine, or an analogue of any of the foregoing. In certain embodiments, the therapeutic combination is a synergistic combination. In certain embodiments, the therapeutic combination further comprises a carbonic anhydrase inhibitor, a potassium supplement, a sodium channel blocker, or any combination thereof. In certain embodiments, the carbonic anhydrase inhibitor is selected from is acetazolamide, topiramate, methazolamide, zonisamide, sulthiame or dichlorphenamide, or an analogue of any of the foregoing. In certain embodiments, the potassium-sparing diuretic is amiloride, eplerenone, spironolactone or triamterene, or an analogue of any of the foregoing. In certain embodiments, the sodium channel blocker is mexiletine or flecainide, or ana analogue of any of the foregoing.

In one aspect, a therapeutic combination is provided comprising a potassium channel opener and a sodium channel blocker. In certain embodiments, the potassium channel opener is retigabine, (10,10-bis(4-pyridinylmethyl)-9(10H)-anthracenone dihydrochloride), N-[4-(6-fluoro-3,4-dihydro-1H-isoquinolin-2-yl)-2,6-dimethylphenyl]-3,3-dimethylbutanamide, (N-[(3-fluorophenyl)-methyl]-1-(2-methoxyethyl)-4-methyl-2-oxo-(7-trifluoromethyl)-1H-quinoline-3-carboxylic acid amide), N-[4-(6-fluoro-3,4-dihydro-1H-isoquinolin-2-yl)-2,6-dimethylphenyl]-3,3-dimethylbutanamide or flupirtine, or an analogue of any of the foregoing. In certain embodiments, the therapeutic combination is a synergistic combination. In certain embodiments, the therapeutic combination further comprises a carbonic anhydrase inhibitor, a potassium supplement, a sodium channel blocker, or any combination thereof. In certain embodiments, the carbonic anhydrase inhibitor is selected from is acetazolamide, topiramate, methazolamide, zonisamide, sulthiame or dichlorphenamide, or an analogue of any of the foregoing. In certain embodiments, the potassium-sparing diuretic is amiloride, eplerenone, spironolactone or triamterene, or an analogue of any of the foregoing. In certain embodiments, the sodium channel blocker is mexiletine or flecainide, or an analogue of any of the foregoing.

In one aspect, a therapeutic combination is provided comprising a potassium channel opener, a carbonic anhydrase inhibitor, a potassium supplement and a potassium-sparing diuretic.

In one aspect, a method is provided for preventing or treating a skeletal muscle disease in a subject comprising administering to the subject an effective amount of a therapeutic combination of a potassium channel opener and a carbonic anhydrase inhibitor. In certain embodiments, the therapeutic combination is a synergistic combination. In certain embodiments, the skeletal muscle disease is an inherited skeletal muscle disease or a sporadic skeletal muscle disease. In certain embodiments, the inherited skeletal muscle disease is periodic paralysis. In certain embodiments, the periodic paralysis is hypokalemic periodic paralysis, hyperkalemic periodic paralysis, Andersen Tawil syndrome (ATS), or thyrotoxic periodic paralysis. In certain embodiments, the thyrotoxic periodic paralysis is endogenous or caused by exogenous thyroid hormone administration.

In certain embodiments, the potassium channel opener is a Kᵥ7 channel opener. In certain embodiments, the Kᵥ7 channel opener is a KCNQ2/3 (Kᵥ7.2/7.3), KCNQ2, KCNQ3, KCNQ4 or KCNQ5 potassium channel opener. In certain embodiments, the Kᵥ7 channel opener is selected from retigabine, (10,10-bis(4-pyridinylmethyl)-9(10H)-anthracenone dihydrochloride), N-[4-(6-fluoro-3,4-dihydro-1H-isoquinolin-2-yl)-2,6-dimethylphenyl]-3,3-dimethylbutanamide, (N-[(3-fluorophenyl)-methyl]-1-(2-methoxyethyl)-4-methyl-2-oxo-(7-trifluoromethyl)-1H-quinoline-3-carboxylic acid amide), N-[4-(6-fluoro-3,4-dihydro-1H-isoquinolin-2-yl)-2,6-dimethylphenyl]-3,3-dimethylbutanamide or flupirtine, or an analogue of any of the foregoing; and the carbonic anhydrase inhibitor is acetazolamide, topiramate, methazolamide, zonisamide, sulthiame or dichlorphenamide, or an analogue of any of the foregoing.

In certain embodiments, the effective amount comprises administering the potassium channel opener from between about 30 minutes prior to consuming food until 12 hours after consuming food.

In certain embodiments, the preventing or treating is abortive therapy. In certain embodiments, the abortive therapy is achieved with a single dose of potassium channel opener and single dose of carbonic anhydrase inhibitor. In certain embodiments, the potassium channel opener is retigabine, (10,10-bis(4-pyridinylmethyl)-9(10H)-anthracenone dihydrochloride), N-[4-(6-fluoro-3,4-dihydro-1H-isoquinolin-2-yl)-2,6-dimethylphenyl]-3,3-dimethylbutanamide, (N-[(3-fluorophenyl)-methyl]-1-(2-methoxyethyl)-4-methyl-2-oxo-(7-trifluoromethyl)-1H-quinoline-3-carboxylic acid amide), N-[4-(6-fluoro-3,4-dihydro-1H-isoquinolin-2-yl)-2,6-dimethylphenyl]-3,3-dimethylbutanamide or flupirtine, or an analogue of any of the foregoing; and the carbonic anhydrase inhibitor is acetazolamide, topiramate, methazolamide, zonisamide, sulthiame or dichlorphenamide, or an analogue of any of the foregoing.

In certain embodiments, the preventing or treating therapy is prophylactic therapy. In certain embodiments, the potassium channel opener is retigabine, (10,10-bis(4-pyridinylmethyl)-9(10H)-anthracenone dihydrochloride), N-[4-(6-fluoro-3,4-dihydro-1H-isoquinolin-2-yl)-2,6-dimethylphenyl]-3,3-dimethylbutanamide, (N-[(3-fluorophenyl)-methyl]-1-(2-methoxyethyl)-4-methyl-2-oxo-(7-trifluoromethyl)-1H-quinoline-3-carboxylic acid amide), N-[4-(6-fluoro-3,4-dihydro-1H-isoquinolin-2-yl)-2,6-dimethylphenyl]-3,3-dimethylbutanamide or flupirtine, or an analogue of any of the foregoing, and the carbonic anhydrase inhibitor is acetazolamide, topiramate, methazolamide, zonisamide, sulthiame or dichlorphenamide, or an analogue of any of the foregoing.

In any of the foregoing methods, in some embodiments, provided is further administering to the subject a potassium-sparing diuretic, a potassium supplement, a sodium channel blocker, or any combination thereof. In certain embodiments, the potassium-sparing diuretic is amiloride, eplerenone, spironolactone or triamterene. In certain embodiments, the sodium channel blocker is mexiletine or flecainide.

In certain embodiments, the subject is administered a potassium channel opener, a carbonic anhydrase inhibitor, a potassium supplement and a potassium sparing diuretic.

In certain embodiments, the skeletal muscle disease is hyperkalemic periodic paralysis. In certain embodiments, the subject is administered a potassium channel opener, a carbonic anhydrase inhibitor, a potassium supplement, a potassium sparing diuretic and a sodium channel blocker.

In any of the foregoing embodiments, treating or preventing is hastening recovery from an episode, aborting an episode, reducing severity of a focal attack, reducing duration of an episode, preventing loss of muscle force, ameliorating an episode of weakness, or any combination thereof.

### BRIEF DESCRIPTIONS OF THE DRAWINGS

FIGS. 1A-1C. Ex vivo contraction test for susceptibility to HypoPP. FIG. 1A: Isometric force measured in response to tetanic stimulation of the soleus muscle from the Naᵥ1.4-R669H mouse. The left panel shows the baseline force during an isometric contraction in 4.7 mM K⁺, and then a decrease in the peak force 4 min after bath exchange to 2 mM K⁺. In a bath containing 10 mM retigabine (right panel), the baseline force in 4.7 mM K⁺ was unchanged and the peak force in preserved during a 2 mM K⁺ challenge. FIG. 1B: The time course for the change in relative peak force in response to a 2 mM K⁺ challenge is shown in control (open symbols, no drug) and after pre-treatment with 10 mM retigabine (filled symbols). These are representative responses from single trials for the soleus muscle in Naᵥ1.4-R669H (left panel, solid circles) and the EDL in Caᵥ1.1-R528H (right). For each trial, the internal control (no drug) is the response measured in the contralateral muscle from the same animal. FIG. 1C: A tetanic contraction of the soleus muscle was recorded every 2 min, and the peak force was normalized to the average value over the first 5 measurements in 4.7 mM K⁺. During a 30 min challenge with 2 mM K⁺ (40 min to 70 min) the peak force waxed and waned in an oscillatory pattern with a nadir of 40% loss (black trace). Pre-treatment with 2 μM retigabine markedly attenuated the loss of peak force.

FIG. 2. Protocol to quantify protection from loss of force in a low K⁺ challenge, when the response waxes and wanes in an oscillatory pattern for the soleus muscle from Naᵥ1.4-R669H. This representative contraction test shows variability in the extent of protection by retigabine; compare the small difference in control to retigabine nadirs at 24 min. versus the larger difference at 42 min. In general, the protection in 1 mM retigabine was less than for 10 mM (FIG. 1). The extent of protection was measured at each nadir (generally two within the 30 min low-K⁺ challenge) as the ratio of the loss of force in retigabine (distance “a”) to the loss in control with no drug (distance “b”). The average of these two measurements was used as the quantitative estimate for the extent of protection for that animal. The low-K⁺ challenge (2 mM) was applied from 20 to 50 min.

FIG. 3. Dose-Response relation for the protection by retigabine and by pinacidil. Each symbol is a mean value±SEM (n=2 to 4) for the relative protection of force in pair-wise comparisons of drug to no drug for a challenge with 2 mM K⁺ (same mouse, two different muscles from either hindlimb). The curves show fitted estimates for a single binding-site model with K_{d} values of 0.82±0.13 mM (Naᵥ1.4-R669H) and 1.9±0.48 mM (Caᵥ1.1-R528H) for retigabine and 8.1±0.49 mM for pinacidil (Naᵥ1.4-R669H). The therapeutic range for unbound drug concentration is shown by the shaded region.

FIGS. 4A-4B. Retigabine promotes recovery from the loss of force for HypoPP muscle while in low K⁺. Examples of retigabine-induced rescue from loss of force are shown by comparison to the control response in the contralateral muscle. FIG. 4A: For the soleus muscle from the Naᵥ1.4-R669H mouse, application of 10 mM retigabine promptly aborted the oscillatory loss of force when applied either 10 min (left) or 30 min (right) after the onset of 2 mM K⁺. Similar responses were observed in 5 of 6 trials to test enhanced recovery by 10 mM retigabine. FIG. 4B: For the EDL muscle from the Caᵥ1.1-R528H mouse, 10 mM retigabine also induced a recovery of relative peak force compared to the response from the contralateral control. For both the soleus and the EDL muscles, recovery of relative peak force to the sustained value of 0.83 is comparable to the relative force for WT soleus in 2 mM K⁺.

FIGS. 5A-5B. Retigabine prevents the paradoxical depolarization of HypoPP fibers during a low K⁺ challenge. Traces show continuous recordings of the resting potential, measured by microelectrode impalement of a fiber in an ex vivo mount of the EDL muscle. FIG. 5A: In WT EDL, low K⁺ challenges of 2 mM or 1 mM elicited hyperpolarization (grey shaded regions) and an extremely low K⁺ of 0.5 mM was required to induce paradoxical depolarization (magenta region). Repolarization occurred upon turn to 4.7 mM K⁺ (arrow). FIG. 5B: For Naᵥ1.4-R669H fibers, paradoxical depolarization occurred for more modest reduction of K⁺ to 1.0 mM or 2.0 mM (magenta regions). Application of retigabine before the low K⁺ challenges prevented the paradoxical depolarization (hatched regions labeled protection). After paradoxical depolarization was established with a low K⁺ challenge, application of retigabine induced fiber repolarization (hatched regions labeled rescue).

FIG. 6. The protective effect of retigabine was prevented by the Kᵥ7 channel inhibitor XE991. Paired soleus muscles from the same HypoPP mouse were used to test for an effect of XE991. Application of XE991 to one muscle did not alter the baseline peak tetanic force (10 min., magenta symbols). Pretreatment with retigabine at 20 min (solid circles) prevented the loss of force during a low-K⁺ challenge (40 to 60 min.). In contrast, an exceptionally large loss of force occurred for the XE991-exposed soleus muscle, despite the same pretreatment with retigabine.

FIG. 7 shows that retigabine is effective a preventing the loss of muscle force that occurs with a low K challenge in a pharmacological model of Andersen-Tawil Syndrome. The pharmacological model of ATS is created with a low concentration of barium (50 μM), which blocks Kir potassium channels. Kir2.1 is deficient in ATS.

FIG. 8 shows that retigabine is partially effective in preventing the loss of force in the mouse model of hyperkalemic periodic paralysis (HyperPP).

FIG. 9 shows that the KV7 channel opener XEN1101 provides complete protection from the loss of force in our NaV1.4-R669H mouse model of HypoPP.

### DETAILED DESCRIPTION

The present disclosure provides effective methods for the prevention and treatment of a skeletal muscle disease by administering to a subject a Kᵥ7 channel opener. Prevention and treatment comprise, by way of non-limiting examples, reducing the severity and/or duration of episodes of skeletal muscle weakness, reducing the frequency of occurrence of episodes of skeletal muscle weakness, preventing the occurrence of episodes of skeletal muscle weakness, accelerating recovery from an episode of skeletal muscle weakness, aborting an imminent or ongoing episode of skeletal muscle weakness, and any combination of any of the foregoing.

Heretofore, interest in targeting the Kᵥ7 channel was confined to the heart and neurons. The recognition of a role for Kᵥ7 in skeletal muscle as disclosed herein and thus prevention or amelioration of conditions and diseases in which targeting skeletal muscle Kᵥ7 is salutary, is provided as described herein. As will be seen in the examples herein, the efficacy of one non-limiting example of a Kᵥ7 channel opener, retigabine, was evaluated and shown to prevent the loss of force and to hasten recovery of force in a mouse model of hypokalemic periodic paralysis (HypoPP). Thus, in one embodiment, the present disclosure provides effective methods for the prevention and treatment of periodic paralysis by administering to a subject a Kᵥ7 channel opener. As described herein, such administering is provided to prevent, abort, reduce, shorten, ameliorate and/or provide benefit to a subject at risk for, diagnosed with, or otherwise affected by a skeletal muscle disease such as but not limited to periodic paralysis.

In other embodiments, various forms of periodic paralysis, as well as other skeletal muscle diseases, are amenable to such methods of treatment.

### Skeletal Muscle Diseases

The methods described herein are applicable to various skeletal muscle conditions and diseases in which targeting the Kᵥ7 potassium channel is salutary. Such conditions and diseases include, but are not limited to, inherited (genetic) and non-inherited (non-genetic) conditions and diseases. In one embodiment, the skeletal muscle disease is a skeletal muscle channelopathy. In one embodiment the skeletal muscle channelopathy is a skeletal muscle potassium channel channelopathy. In one embodiment, the skeletal muscle disease is a periodic paralysis (described in more detail below).

### Periodic Paralysis

The methods described herein are applicable to various forms of periodic paralysis. In one embodiment, the period paralysis is hypokalemic period paralysis (hypoPP). In one embodiment, the periodic paralysis is hyperkalemic period paralysis. In one embodiment the periodic paralysis is Andersen Tawil syndrome (ATS). In one embodiment, the period paralysis is thyrotoxic periodic paralysis.

In some embodiments, methods described herein prevent loss of muscle force. In some embodiments, methods described herein prevent loss of muscle force in low potassium. In some embodiments, methods described herein prevent loss of muscle force in hypoPP.

In some embodiments, methods described herein prevent loss of muscle force in low potassium in hypoPP. In some embodiments, methods described herein prevent loss of muscle force in hyperPP. In some embodiments, methods described herein prevent loss of muscle force in ATS. In some embodiments, methods described herein prevent loss of muscle force in low potassium in ATS.

In some embodiments, methods described herein prevent, e.g., prophylax, episodes of weakness in periodic paralysis. In some embodiments, prophylaxis of episodes of periodic paralysis in hypoPP, hyperPP, ATS and/or thyrotoxic periodic paralysis are provided.

In some embodiments, methods described herein hasten recovery from periodic paralysis.

In some embodiments, methods described herein reduce severity and/or duration of an episode of weakness in periodic paralysis.

In some embodiments, methods described herein provide abortive therapy for periodic paralysis.

In some embodiments, methods described herein reduced the frequency of occurrence of episodes of periodic paralysis.

Any of the foregoing embodiments are applicable to episodes of, or chronic muscle weakness in, skeletal muscle diseases, such as any of those described herein.

### Assessing Effectiveness

In some embodiments, methods described herein reduce the severity a skeletal muscle disease such as periodic paralysis. In one embodiment, measuring severity comprises measuring CMAP amplitude expressed as a percent of peak CMAP during or after the McManis exercise.

In some embodiments, methods described herein reduce the duration of an episode of muscle weakness in a skeletal muscle disease such as periodic paralysis. In one embodiment, measuring duration comprises determining the time between treatment administration until CMAP returns to 65% of peak CMAP within 4 hours following the treatment intake.

In some embodiments, methods described herein reduce the severity a focal attack within two hours of treatment in a skeletal muscle disease such as periodic paralysis. In one embodiment, measuring severity comprises measuring CMAP amplitude (in percent compared to peak) area under the curve (AUC) from treatment administration until two hours post-treatment.

In some embodiments, methods described herein reduce the severity a focal attack within two to four hours post-treatment in a skeletal muscle disease such as periodic paralysis. In one embodiment, measuring severity comprises measuring CMAP amplitude (in percent compared to peak) area under the curve (AUC) from treatment administration during the third and fourth hours post-treatment.

In some embodiments, methods described herein prevent loss of muscle force in a skeletal muscle disease such as periodic paralysis. In one embodiment, measuring prevention of loss of muscle force is determined by manual muscle testing or by dynamometry.

In some embodiments, methods described herein ameliorate an episode of weakness in a skeletal muscle disease. In some embodiments, methods described herein ameliorate an episode of weakness in periodic paralysis. In some embodiments, methods described herein ameliorate an episode of weakness in hypokalemic periodic paralysis.

In one embodiment, measuring amelioration of an episode of weakness is determined by the ability to raise limbs against gravity, to rise from a sitting position, and to walk without assistance.

In some embodiments, methods described herein hasten the recovery from an episode of weakness in a skeletal muscle disease. In some embodiments, methods described herein hasten the recovery from an episode of weakness in periodic paralysis. In some embodiments, methods described herein hasten the recovery from an episode of weakness in hypokalemic periodic paralysis.

In some embodiments, methods described herein hasten the recovery from an episode of muscle weakness in a skeletal muscle disease such as periodic paralysis. In one embodiment, measuring hastened recovery comprises determining the time between treatment administration until CMAP returns to 65% of peak CMAP following the treatment intake.

In some embodiments, the methods described herein abort an episode of a skeletal muscle disease. In some embodiments, the methods described herein abort an episode of periodic paralysis. In some embodiments, the methods described herein abort an episode of hypokalemic periodic paralysis.

In one embodiment, measuring aborting an episode of a skeletal muscle disease is determined by the time interval between the administration of therapy and regaining the ability to raise limbs against gravity, sit independently, and/or ambulate without assistance.

In some embodiments, the methods provided herein comprise administering to a subject at risk for, or having a sequela of, a skeletal muscle disease an effective amount of a Kv7 channel opener, to achieve any of the aforementioned purposes.

### Kv7 Channel Openers

The methods disclosed herein provide for the use of a Kv7 channel opener (also called a Kv7 channel activator or Kv7 channel agonist) for the prevention or treatment of various skeletal muscle diseases and conditions.

Retigabine. In one embodiment, the Kv7 channel opener is retigabine. Retigabine (egozabine; D-23129; XEN496; ethyl [2-amino-4-[[(4-fluorophenyl)methyl]amino]phenyl]carbamate; CAS Registry No. 150812-12-7) is a selective agonist of the Kv7 family of potassium channels. It was marketed as an anticonvulsant for the treatment of epilepsy under the names of POTIGA (US) and TROBALT (EMA) but discontinued because of side effects including skin discoloration and eye abnormalities; other side effects include tremor, memory loss, gait disturbances and double vision.

When it was marketed, the dosing regimen for approved indications of adjunctive therapy in partial onset seizures uncontrolled by current medications and for whom the benefits outweigh the risk of retinal abnormalities and potential decline in visual acuity, the initial dose is 100 mg PO q8 hr; may increase dose at weekly intervals, not to exceed dosage increase of 150 mg/day/week. An optimized effective dosage is between 200 mg 3 times daily (600 mg per day) to 400 mg 3 times daily (1,200 mg per day).

### Other Kv7 Channel Openers

Some examples of Kv7 channel openers (also called Kv7 channel activator or Kv7 channel agonist) are provided below, but the methods of use described herein are not so limited and the methods disclosed herein comprise use of any Kv7 channel opener. The present disclosure encompasses such herein described Kv7 channel openers merely as non-limiting examples, and any such compounds and analogues thereof are embraced herein.

Non-limiting examples of retigabine analogues useful for the purposes described herein are described in Ostacolo et al., J Med Chem 2020, 63, 1, 163-185.

XE991 (10,10-bis(4-Pyridinylmethyl)-9(10H)-anthracenone dihydrochloride) is described in Wang et al (Science. 1998 Dec. 4; 282(5395):1890-3) KCNQ2 and KCNQ3 potassium channel subunits: molecular correlates of the M-channel. Science 282 1890 and Zaczek et al (1998) Two new potent neurotransmitter release enhancers, 10,10-bis(4-pyridinylmethyl)-9(10H)-anthracenone and 10,10-bis(2-fluoro-4-pyridinylmethyl)-9(10H)-anthracenone: comparison to linopirdine. J. Pharmacol. Exp. Ther. 285 724.

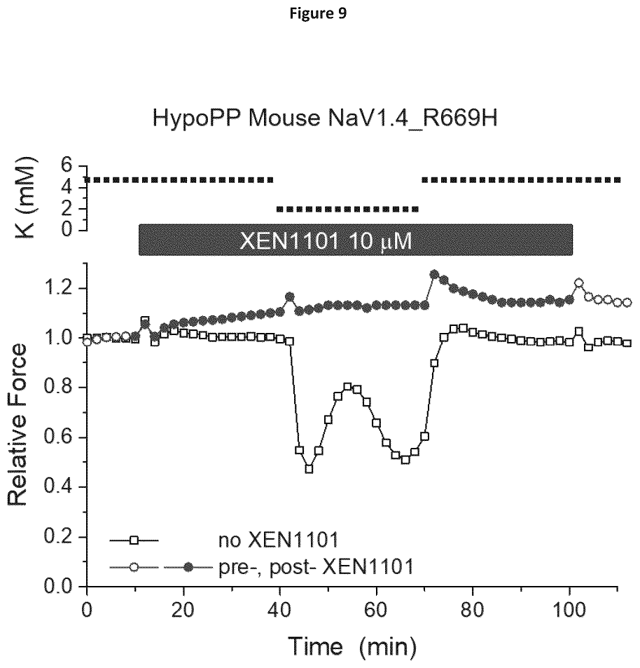

XEN1101, N-[4-(6-fluoro-3,4-dihydro-1H-isoquinolin-2-yl)-2,6-dimethylphenyl]-3,3-dimethylbutanamide (Encukalner), is a Kv7 channel opener being developed for epilepsy and major depressive disorder. As shown in Example 5 (FIG. 9), XEN1101 provides complete protection from the loss of force in the NaV1.4-R669H mouse model of HypoPP.

XEN496 is a synonym for retigabine (ezogabine).

GRT-X ((N-[(3-fluorophenyl)-methyl]-1-(2-methoxyethyl)-4-methyl-2-oxo-(7-trifluoromethyl)-1H-quinoline-3-carboxylic acid amide) is a Kv7 channel activator and TSPO (translocator protein) activator.

Other Kv7 channel openers are described in U.S. Pat. No. 8,993,593, such as N-[4-(6-fluoro-3,4-dihydro-1H-isoquinolin-2-yl)-2,6-dimethylphenyl]-3,3-dimethylbutanamide.

Other Kv7 channel openers are described in U.S. Pat. No. 8,293,911, such as N-(2-chloro-4-(3,4-dihydroisoquinolin-2(1H)-yl)-6-(trifluoromethyl)phenyl)-3,3-dimethylbutanamide; N-(4-(3,4-dihydroisoquinolin-2(1H)-yl)-2,6-dimethylphenyl)-3,3-dimethylbutanamide; N-(2-chloro-4-(3,4-dihydroisoquinolin-2(1H)-yl)-6-(trifluoromethyl)phenyl)-3-cyclopentylpropanamide; N-(2-chloro-4-(6-fluoro-3,4-dihydroisoquinolin-2(1H)-yl)-6-(trifluoromethylphenyl)-3,3-dimethylbutanamide; N-[2-chloro-4-(3,4-dihydro-1H-isoquinolin-2-yl)-6-methyl phenyl]-3,3-dimethylbutanamide; N-[2-chloro-4-(6-fluoro-3,4-dihydro-1H-isoquinolin-2-yl)-6-trifluoromethyl phenyl]-3-cyclopentylpropionamide; N-[2,6-dimethyl-4-(6-trifluoromethyl-3,4-dihydro-1H-isoquinolin-2-yl)-phenyl]-3,3-dimethylbutanamide; N-[2-chloro-6-trifluoromethyl-4-(6-trifluoromethyl-3,4-dihydro-1H-isoquinolin-2-yl)-phenyl]-3,3-dimethylbutanamide; N-[2-chloro-4-(6-chloro-3,4-dihydro-1H-isoquinolin-2-yl)-6-trifluoromethyl phenyl]-3,3-dimethylbutanamide; N-[4-(6-chloro-3,4-dihydro-1H-isoquinolin-2-yl)-2,6-dimethyl-phenyl]-3,3-dimethylbutanamide; N-[4-(6-fluoro-3,4-dihydro-1H-isoquinolin-2-yl)-2,6-dimethyl phenyl]-3,3-dimethylbutanamide; N-[2-chloro-4-(7-fluoro-3,4-dihydro-1H-isoquinolin-2-yl)-6-trifluoromethyl-phenyl]-3,3-dimethylbutanamide; N-[4-(7-fluoro-3,4-dihydro-1H-isoquinolin-2-yl)-2,6-dimethyl-phenyl]-3,3-dimethylbutanamide; N-[2-chloro-4-(6-fluoro-3,4-dihydro-1H-isoquinolin-2-yl)-6-methylphenyl]-3,3-dimethylbutanamide; N-[2-chloro-4-(7-fluoro-3,4-dihydro-1H-isoquinolin-2-yl)-6-methylphenyl]-3,3-dimethylbutanamide; N-[2-chloro-6-methyl-4-(6-trifluoromethyl-3,4-dihydro-1H-isoquinolin-2-yl)-phenyl]-3,3-dimethylbutanamide; N-[2-chloro-4-(6-chloro-3,4-dihydro-1H-isoquinolin-2-yl)-6-methyl-phenyl]-3,3-dimethylbutanamide; N-[2-chloro-4-(6-fluoro-3,4-dihydro-1H-isoquinolin-2-yl)-phenyl]-3,3-dimethylbutanamide; N-[4-(6-fluoro-3,4-dihydro-1H-isoquinolin-2-yl)-2-methyl-phenyl]-3,3-dimethylbutanamide; N-[4-(6-fluoro-3,4-dihydro-1H-isoquinolin-2-yl)-2-trifluoromethylphenyl]-3,3-dimethylbutanamide; N-[2-chloro-4-(6-trifluoromethyl-3,4-dihydro-1H-isoquinolin-2-yl)-phenyl]-3,3-dimethylbutanamide; N-[4-(7-fluoro-3,4-dihydro-1H-isoquinolin-2-yl)-2-trifluoromethyl-phenyl]-3,3-dimethylbutanamide; 3,3-dimethyl-N-[2-trifluoromethyl-4-(7-trifluoromethyl-3,4-dihydro-1H-isoquinolin-2-yl)-phenyl]butanamide; N-[4-(6-methoxy-3,4-dihydro-1H-isoquinolin-2-yl)-2,6-dimethyl-phenyl]-3,3-dimethylbutanamide; N-[4-(3,4-dihydro-1H-isoquinolin-2-yl)-2-methoxy-6-methyl-phenyl]-3,3-dimethylbutanamide; N-[2-chloro-4-(3,4-dihydro-1H-isoquinolin-2-yl)-6-trifluoromethoxy-phenyl]-3,3-dimethylbutanamide; N-[4-(3,4-dihydro-M-isoquinolin-2-yl)-2,6-dimethoxy-phenyl]-3,3-dimethylbutanamide; N-[2,6-dimethyl-4-(7-trifluoromethyl-3,4-dihydro-1H-isoquinolin-2-yl)-phenyl]-3,3-dimethyl butanamide; N-[2,6-Dimethyl-4-(6-trifluoromethyl-3,4-dihydro-1H-isoquinolin-2-yl)-phenyl]-3,3-dimethyl-thiobutanamide; [2,6-Dimethyl-4-(6-trifluoromethyl-3,4-dihydro-1H-isoquinolin-2-yl)-phenyl]-carbamic acid ethyl ester; and N-[2,6-Dimethyl-4-(7-trifluoromethyl-3,4-dihydro-1H-isoquinolin-2-yl)-phenyl]-3,3-dimethyl butanamide.

Other Kv7 channel openers useful for the purposes disclosed herein are described in Seefeld et al., Bioorganic & Medicinal Chemistry Letters 2018, 28:3793-3797, such as PF05020182, ICA-27243; such disclosed compounds in Seefeld et al. are incorporated herein by reference.

Flupirtine (carbamic acid, N-[2-amino-6-[[(4-fluorophenyl)methyl]amino]-3-pyridinyl]-, ethyl ester) is a non-opioid analgesic with neuronal potassium channel opener activity.

Any of the references cited here are incorporated herein by reference in their entireties.

### Methods of Treatment

The methods disclosed herein provide for the use of a Kv7 channel opener for the prevention or treatment of symptoms or sequelae of various skeletal muscle diseases and conditions, and their symptoms, for prophylactic treatment, for chronic treatment, for sporadic treatment, for episodic treatment, among others. Such symptoms or sequelae include but are not limited to chronic or periodic muscle weakness. The Kv7 channel opener may be administered chronically, administered for a specific duration, or administered when needed for example when a sign or symptom arises in the subject, such as an episode of weakness or paralysis in hypoPP. The Kv7 channel opener may be administered to hasten recovery from an attack of weakness or paralysis, wherein the underlying diseases is being treated or not being treated with a Kv7 channel opener, or by any other treatment. In one embodiment, the sporadic or episodic treatment with a Kv7 channel opener is of a higher and/or more frequent dose level and/or dosing regimen than administered for prophylactic or preventative use. Such methods are similarly applicable to treatment of hyperPP.

### Dosage Regimen Selection

The selection of the efficacious dose and other aspects of the dosing regimen, such as frequency of dosing, duration of dosing, etc.) will be guided by, and readily determined by, the medical professional based on the characteristic of the Kv7 channel opener and the subject, among other factors. In one embodiment, the pharmacokinetics of the Kv7 channel opener will guide the dose level and frequency of dosing (see, for example, Ferron et al., Multiple-dose, linear, dose-proportional pharmacokinetics of retigabine in healthy volunteers, 2002, J Clin Pharm 42:175-182) for guidance on one Kv7 channel opener.

In some embodiments, a dose of retigabine for prophylaxis of periodic paralysis is about 600 mg/day. In some embodiments, the dose is 1200 mg/day (400 mg q8h). In some embodiments, a dose of retigabine to abort an imminent episode is 200 mg. In one embodiment, a dose of retigabine to abort an imminent episode is 400 mg.

In some embodiments, an effective regimen is administering the Kv7 potassium channel opener from between about 30 minutes prior to consuming food until 12 hours after consuming food.

Effectiveness of a particular dosing regimen may be assessed as described herein above.

The dosing regimen used for the aforementioned purposes may be titrated to achieve optimal effectiveness and minimal side effects. In one non-limiting example, retigabine for prophylactic treatment of hypoPP may be administered starting at 100 mg PO q8 hr (every 8 hours); which dosing regimen may increase dose at weekly intervals, not to exceed a dosage increase of 150 mg/day/week.

In other embodiments, the prophylactic dose is 150 mg, 200 mg, 250 mg, 300 mg, 350 mg, 400 mg, 450 mg or 500 mg, administered daily, twice daily, every 4 hours, or every 8 hours. In some embodiments, the preventive, sporadic or episodic dose, for use for example for the purposes disclosed herein above, may be 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg or 100 mg administered once, or daily, twice daily, every 4 hours, or every 8 hours., for the duration required for such uses. For any such uses, the dose may be escalated over time, such as but not limited to an increase in 10 mg, 25 mg, 50 mg, 100 mg or 150 mg/week. Titration of the dose and dosing regimen for a particular patient or purpose, and escalation thereof, is embraced herein. The health care professional will be readily guided by safety and desired efficacy in selecting the dose and dosing regimen, and the escalation thereof.

Various routes of administration are embodied herein, such as but not limited to oral, nasal or parenteral administration. Depending upon the treatment need, a dose of a Kv7 channel opener needed to abort an episode may be given by injection (e.g., subcutaneous or intramuscular), by nasal administration, or by oral administration (including sublingual). For prophylaxis, an oral dosing regimen is provided.

### Unit Dose

A unit dose of a Kv7 channel opener as described herein for the purposes disclosed is dependent upon the pharmacokinetics among other factors. For retigabine, for example, a unit dose is provided at 50 mg, 200 mg, 300 mg or 400 mg.

Therapeutic Combinations with a Kv7 Channel Opener

In some embodiments, a dosing regimen for treatment or prevention of a disease or condition, or symptom or sequela, of skeletal muscle described herein is achieved with a combination of a Kv7 channel opener and one or more other agents that provide an efficacious treatment for a subject. Such treatment includes prophylaxis or treatment of hypoPP, hyperPP, ATS and/or thyrotoxic periodic paralysis. As will be noted here, in some embodiments the combination is synergistic, such that a combination of sub-efficacious doses of either component are together more effective than the combined efficacy of each administered alone. For example, in one embodiment, a less than fully effective dose of a KV7 channel opener such as retigabine or XEN1101 in combination with a carbonic anhydrase inhibitor may provide superior efficacy than achieved by the lower dose of KV7 channel opener or the carbonic anhydrase inhibitor as separate regimens in hypoPP. In another non-limiting example, as noted in the examples herein, while in one model retigabine is partially effective in preventing the loss of force in a mouse model of hyperkalemic periodic paralysis, a therapeutic combination with another component such as a carbonic anhydrase inhibitor may provide synergy.

In one embodiment, the therapeutic combination is a Kv7 channel opener and a carbonic anhydrase inhibitor. Non-limiting examples of carbonic anhydrase inhibitors include acetazolamide, topiramate, methazolamide, zonisamide, sulthiame and dichlorphenamide, or an analogue of any of the foregoing.

In one embodiment, the therapeutic combination is a Kv7 channel opener and a potassium-sparing diuretic such as amiloride, eplerenone, spironolactone or triamterene, or an analogue of any of the foregoing.

In one embodiment, the therapeutic combination is a Kv7 channel opener and a potassium supplement.

In one embodiment, the therapeutic combination is a Kv7 channel opener and a sodium channel blocker. In some embodiments, a sodium channel blocker is used in a subject with hyperkalemic periodic paralysis. Non-limiting examples of sodium channel blockers include mexiletine and flecainide, or an analogue of any of the foregoing.

Thus, in some embodiments, the therapeutic combination is a Kv7 channel opener, a potassium supplement and a potassium sparing diuretic. In some embodiments, the therapeutic combination is a Kv7 channel opener, a carbonic anhydrase inhibitor and a potassium supplement. In some embodiments, the therapeutic combination is a Kv7 channel opener, a carbonic anhydrase inhibitor and a potassium sparing diuretic. In one embodiment, the therapeutic combination is a Kv7 channel opener, a carbonic anhydrase inhibitor and a sodium channel blocker. In one embodiment, the therapeutic combination is a Kv7 channel opener, a potassium-sparing diuretic and a sodium channel blocker. In one embodiment, the therapeutic combination is a Kv7 channel opener, a potassium supplement and a sodium channel blocker.

Thus, in some embodiments, the therapeutic combination is a Kv7 channel opener, a potassium supplement, a potassium sparing diuretic and a sodium channel blocker. In some embodiments, the therapeutic combination is a Kv7 channel opener, a carbonic anhydrase inhibitor, a potassium supplement and a sodium channel blocker. In some embodiments, the therapeutic combination is a Kv7 channel opener, a carbonic anhydrase inhibitor, a potassium sparing diuretic and a sodium channel blocker.

In one embodiment, the therapeutic combination is a Kv7 channel opener, a carbonic anhydrase inhibitor, a potassium supplement, a sodium channel blocker and a sodium channel blocker.

In some embodiments, the therapeutic combination is a synergistic combination, for example, the effectiveness achieved by administration of the disclosed combination is greater than the combined effects of the individual or any sub-combinations of components administered at the doses or dose regimens in the combination but in the absence of any one other component.

Thus, in one embodiment, a therapeutic combination of a Kv7 channel opener such as but not limited to those described herein, and a carbonic anhydrase inhibitor such as but not limited to those described herein, elicits greater effectiveness than achieved by either the Kv7 channel opener administered alone or the carbonic anhydrase inhibitor when administered alone.

In one embodiment, a therapeutic combination comprises retigabine and acetazolamide.

Thus, in one embodiment, a therapeutic combination of a Kv7 channel opener such as but not limited to those described herein, a carbonic anhydrase inhibitor such as but not limited to those described herein, a potassium supplement and a potassium sparing diuretic elicits greater effectiveness than achieved by either the Kv7 channel opener administered alone or the carbonic anhydrase inhibitor, potassium supplement or potassium sparing diuretic when administered alone.

In one embodiment, a therapeutic combination comprises retigabine, acetazolamide, a potassium supplement and a potassium sparing diuretic.

Thus, in one embodiment, a therapeutic combination of a Kv7 channel opener such as but not limited to those described herein, a carbonic anhydrase inhibitor such as but not limited to those described herein, a potassium supplement, a potassium sparing diuretic and a sodium channel blocker elicits greater effectiveness than achieved by either the Kv7 channel opener administered alone or the carbonic anhydrase inhibitor, potassium supplement, potassium sparing diuretic or sodium channel blocker when administered alone.

In one embodiment, a therapeutic combination comprises retigabine, acetazolamide, a potassium supplement, a potassium sparing diuretic and a sodium channel blocker.

In any of the foregoing embodiments, any combination including a sodium channel blocker is used in a subject with hyperkalemic periodic paralysis.

### Therapeutic Combination Dosage Regimen Selection

As noted above with regard to the dose and dose regimen of the Kv7 channel opener for any among the various purposes and uses herein disclosed for prophylaxis or treatment of hypoPP, hyperPP, ATS and/or thyrotoxic periodic paralysis, the additional component and any synergistic activity of the combination, the selection of the efficacious dose and other aspects of the dosing regimen, such as for each component the frequency of dosing, duration of dosing, etc.) will be guided by, and readily determined by, the medical professional based on the characteristic of the Kv7 channel opener and the other component of the therapeutic combination, and the subject, among other factors.

Effectiveness of a particular therapeutic combination dosing regimen may be assessed as described herein above.

### Preclinical Studies

As will be seen in the examples below, knock-in mutant mouse models of HypoPP (Scn4a p.R669H, cacnals p.R528H) were used to identify the therapeutic potential of a Kv7 channel opener in this disease with applicability to other skeletal muscle diseases. The mutant mouse model has a robust phenotype with paradoxical fiber depolarization and loss of contractile force in low K+. Susceptibility to HypoPP was determined by an ex vivo assay that measured the loss of force in response to a reduction of extracellular [K+] from 4.7 mM to 2 mM. As will be described, substantial protection from low-K+ induced loss of force was observed after pre-treatment with 1 μM retigabine, and 10 μM completely suppressed any detectable loss of force. By comparison with this same assay, pinacidil, an ATP-sensitive potassium channel opener, was an order of magnitude less potent than retigabine for the prevention of weakness.

Moreover, KV7 channel opener XEN1101 provides complete protection from the loss of force in the NaV1.4-R669H mouse model of HypoPP.

The examples show the KV7-channel opener also hastened recovery in the model. For HypoPP muscle maintained in 2 mM K+ the initial loss of force was reversed by application of retigabine. These data show retigabine, or other KV7 agonists, has the potential to reduce the severity of episodic weakness in HypoPP and is superior to previously studied K+ channel openers acting on KATP channels.

While in principle using a potassium channel opener to increase the membrane conductance to K+ will hyperpolarize the membrane potential toward the Nernst potential for K+(EK≈−95 mV), many criteria must be met for this strategy to be safe and effective. For example, many K+ channels are activated by depolarization, and the drug-induced augmentation of channel opening must occur over an operational voltage range that is relevant for stabilization of a normal resting potential (−80 to −95 mV) and extend to the anomalous depolarization during paralysis (typically −50 to −60 mV). This voltage-dependent property will determine whether the increased K+ conductance will primarily affect the resting potential, the action potential waveform (e.g. amplitude and duration), or both. The compounds including therapeutic combinations disclosed herein achieve these objectives and can be readily adjusted to be effective by one of skill in the art based on the teachings herein.

In some embodiments, the dosing regimen of the compound or therapeutic combination for achieving efficacy as described herein is provided to address the desired effect in skeletal muscle, with recognition of the expression in different tissues of the K+ channels that are activated by the drug; in one embodiment, expression of K+ channels in skeletal muscle provides for the desired effect, with lower expression in other tissues to minimize side effects. The drug potency (small dissociation constant, Kd) and magnitude of the K+ conductance increase are important as well. If the conductance increase is very small, then the drug may not produce a meaningful reduction in the susceptibility to attacks of weakness. Conversely, a very large K+ conductance increase may impair contractility by reducing muscle fiber excitability. Such determination of the appropriate drug and dosing regimen thereof is assessable by the skilled artisan.

Thus, the effectiveness of retigabine at low μM concentrations to protect HypoPP muscle from a loss of force in a low-K+ challenge (2 mM) demonstrates the therapeutic potential of KV7.x channel openers in the symptomatic management of HypoPP.

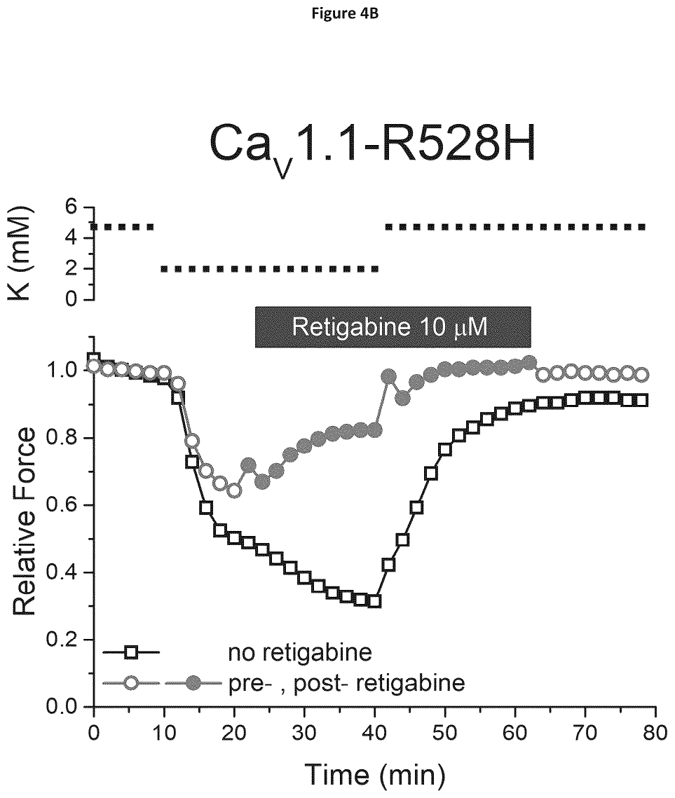

Data provided here showing rescue of contractility (FIG. 4) demonstrates the potential for short-term administration of retigabine, or other KV7 channel openers (non-limiting examples of which are disclosed herein), as abortive therapy for an acute attack of weakness in HypoPP. By way of theory to which applicant is not bound, after a single 100 mg oral dose or retigabine, a Cmax of 390 ng/ml occurs within 1.5 hrs., and the half-life is 8 hrs (Ferron et al., 2002, op. cit.). An unbound retigabine fraction of 0.9 μM will be achieved with a single 400 mg dose (assumes 80% protein binding). Based on this observation, a clinical trial for efficacy of KV7 openers could be performed by assessing the rate of recovery of the compound on muscle action potential in the long exercise test for periodic paralysis. The functional defect in periodic paralysis operates like a binary switch, with a sustained shift between the normal Vrest and an anomalously depolarized value. Consequently, a single dose of retigabine may be sufficient to “reset” Vrest to the normal range and suppress a recurrent attack of weakness over the minutes to hours required for correction of extracellular [K⁺]. An adverse effect of retigabine on skeletal muscle function is unlikely.

### EXAMPLES

### Materials and Methods

Mouse Model of HypoPP. Knock-in mutant mouse models of HypoPP were previously generated; Type 1 with a missense mutation of the Caᵥ1.1 calcium channel (Cacnals p.R528H) and Type 2 with a missense mutation of the Naᵥ1.4 sodium channel (Scn4a p.R663H) homologous to the p.R669H missense mutation in patients with HypoPP Type 2. Mutant mice have a robust HypoPP phenotype with loss of force and paradoxical depolarization of skeletal muscle in response to a challenge in low extracellular [K⁺] (<3 mM) or to intravenous administration of insulin plus glucose. Consistent with the dominant inheritance in patients, heterozygous mutant mice exhibit all the features of HypoPP. Mouse studies have revealed a gene dosage effect of the mutant allele, and homozygous mutant mice were used in this study to improve the sensitivity for detecting a beneficial effect of retigabine on the outcome measure of muscle force.

All procedures performed on mice were in accordance with animal protocols approved by the David Geffen School of Medicine Institutional Animal Care and Use Committee.

Ex vivo Contraction Studies. The outcome measure for susceptibility to HypoPP was the peak isometric force of the soleus muscle, in response to a tetanic stimulation, as previously described. In brief, after euthanizing the animal, the extensor digitorum longus (EDL) muscle or the soleus muscle was dissected free and suspended in a tissue bath held at 37° C. Similar responses were observed for either muscle, but the EDL muscle was preferentially used for Caᵥ1.1-R528H mice because the HypoPP phenotype is more pronounced, and the soleus muscle was used for Naᵥ1.4-R669H mice because the dissection is easier. Muscles were suspended in a bicarbonate-buffered bath that was bubbled continuously with 5% CO₂/95% 02 to maintain a pH of 7.4. The standard bath consisted of (in mM): 118 NaCl, 4.75 KCl, 1.18 MgSO₄, 2.54 CaCl₂), 1.18 NaH₂PO₄, 10 glucose, and 24.8 NaHCO₃. The low-K⁺ solution was identical, except for the reduction of KCl to 2 mM by replacement with NaCl. The osmolality of all solutions was 290 mOsm. Retigabine (gift from Prof. M. Taglialatela, Univ. Naples) and pinacidil (Sigma-Aldrich) were prepared as 100 mM stock solutions in DMSO and diluted into the bath solution for a working concentration of 0.1 to 100 mM. The Kᵥ7 inhibitor, XE991 (Sigma-Aldrich), was prepared in as a 100 mM DMSO stock solution and applied to muscle in a 10 mM bath solution.

Electrical stimulation was applied by a pair of platinum wires, oriented perpendicular to the long axis of the soleus muscle. Suprathreshold stimulation (80 mA) was applied as a tetanic burst (40 pulses, 0.4 msec, at 100 Hz), under computer control. All bath solutions contained 0.25 mM D-tubocurarine (Sigma-Aldrich) to prevent any contribution to muscle excitation from motor nerve endings. Muscle force was measured with a stiff strain gain (Forte 25, World Precision Instruments) and digitally sampled at 5 KHz. Muscle contractility was monitored by measuring the peak isometric force every two minutes, and test solutions were applied by complete exchange of the bath solution.

Membrane Potential Measurements. The membrane potential was recorded by impalement of superficial fibers from an ex vivo whole mount of the EDL muscle. Long-duration stable recording of the resting potential was achieved by using an orthogonal approach to the fiber with fine-tipped microelectrodes (resistance of 15-20 MW, filled with 3 M KCl). The bath solution was the same as that used for the ex vivo contraction studies, continuously bubbled with 5% CO₂/95% O₂, with a flow rate of ˜3 ml/min of the 0.3 ml recording chamber.

The raw data for the outcome measures of this study are digitized traces of muscle force or fiber membrane potential, stored in a custom 16-bit binary format.

### Example 1

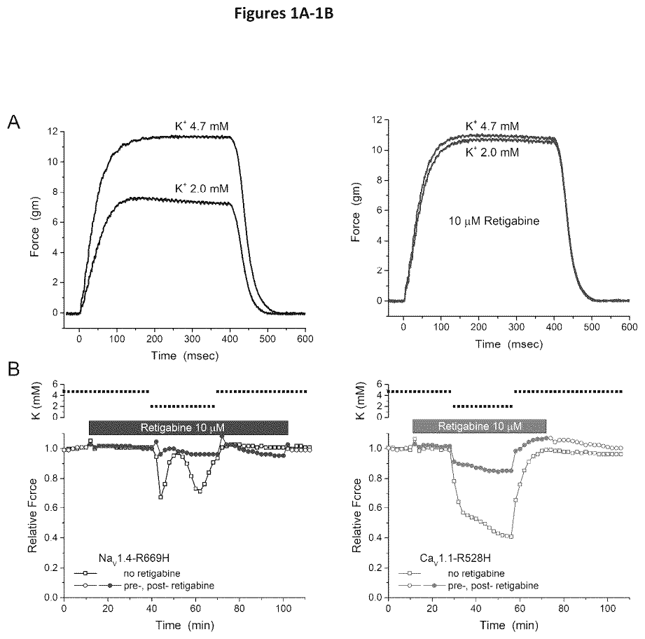

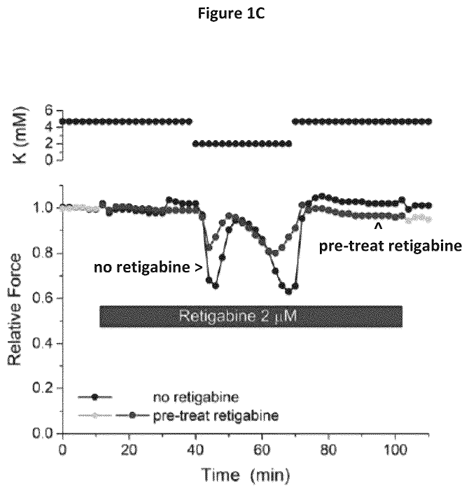

The efficacy of potassium channel openers to prevent the loss of force in HypoPP muscle was assessed by measuring isometric contractions, ex vivo, in both control (4.7 mM) and low extracellular [K⁺] (2.0 mM). The HypoPP phenotype is shown for the drug-free responses of the soleus muscle from an Naᵥ1.4-R669H mouse in the left panel of FIG. 1A. The peak isometric force during the tetanic contraction (100 Hz stimulation) was initially 11.7 gm in control [K⁺], and then decreased to 7.6 gm (35% decrease) after 4 min. in 2 mM [K⁺]. The loss of force is reversible, upon return to 4.7 mM [K⁺] (e.g., see FIG. 1B). For the soleus muscle from the other hindlimb of the same animal, control and low-K⁺ solutions containing 10 mM retigabine were used, and the loss of force from a 2 mM [K⁺] challenge was completely prevented (FIG. 1A, right panel).

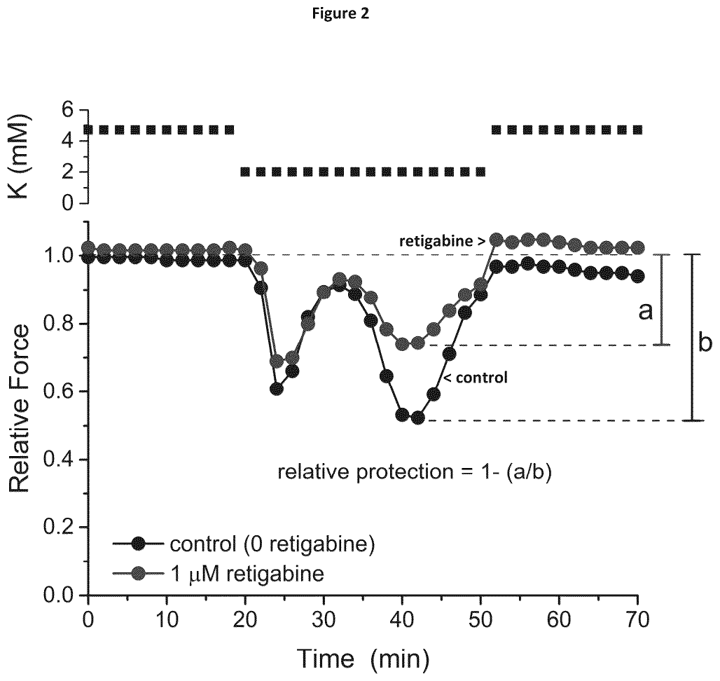

The time course of the relative force in response to a 30 min. low-K⁺ challenge, with and without pretreatment with retigabine, is shown in FIG. 1B. The relative force was stable before the low-K⁺ challenge and was not affected by the addition of retigabine for either the Naᵥ1.4-R669H soleus muscle or the Caᵥ1.1-R528H EDL muscle (time=10 min. in FIG. 1B) over the range of concentrations used (0.1 to 20 mM). As it was previously reported, the loss of force during a low-K⁺ challenge in the Naᵥ1.4-R669H HypoPP mouse in drug-free conditions may exhibit oscillations with spontaneous periods of recovery for the soleus muscle (FIG. 1B, left). While this oscillatory response is variable across different mice, the concordance is high between the left and right soleus muscles of a single mouse, as shown by the in-phase oscillations for control and retigabine in FIG. 2 or for paired drug-free recordings in Wu et al. The representative responses in FIG. 1B reaffirm that pre-treatment with 10 mM retigabine completely suppressed the HypoPP phenotype in the 2 mM [K⁺] challenge, such that the relative force was >0.85 (filled symbols) as occurs for WT muscle.

A method to quantify the extent of protection from loss of force by retigabine is illustrated in FIG. 2. This example was selected to illustrate the maximum variability in the extent of protection for the oscillations of soleus muscle force within a single trial (compare the responses at 24 min to those at 40 min.). Qualitatively, even 1 mM retigabine was sufficient to provide substantial protection. The relative protection was calculated from the responses for each pair of soleus muscles from the same mouse serving as an internal control. In almost every trial (22 out of 24), two clear minima occurred during the 30 min. low-K⁺ challenge. The relative protection for the retigabine response, defined as 1−(force loss in retigabine)/(force loss in control) was calculated for each of the two minima, and the average value was used as the quantitative estimate for the retigabine protection in that trial. Each soleus muscle was used for only a single trial (i.e. control or retigabine) because cumulative effects and run-down may occur if this long protocol (typically 70 min.) was repeated. Oscillations in force did not occur in the low K⁺ challenge for EDL muscle in Caᵥ1.1-R528H mice, and so the time at which the nadir occurred was used to calculate the relative protection.

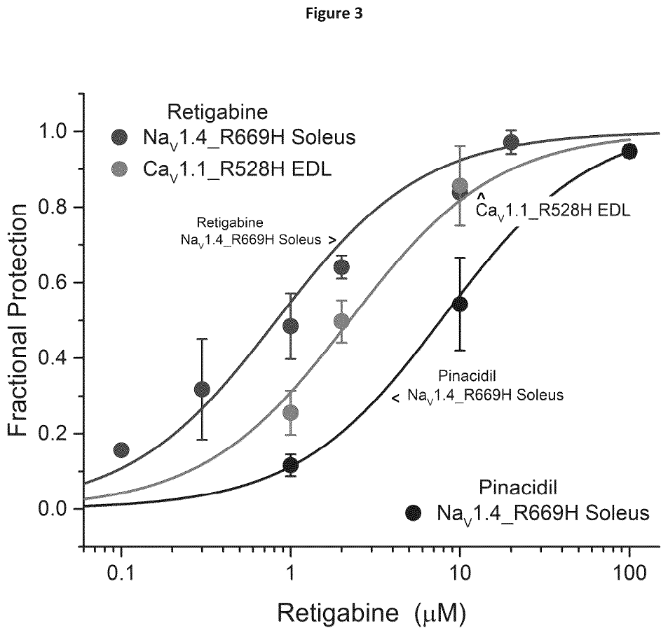

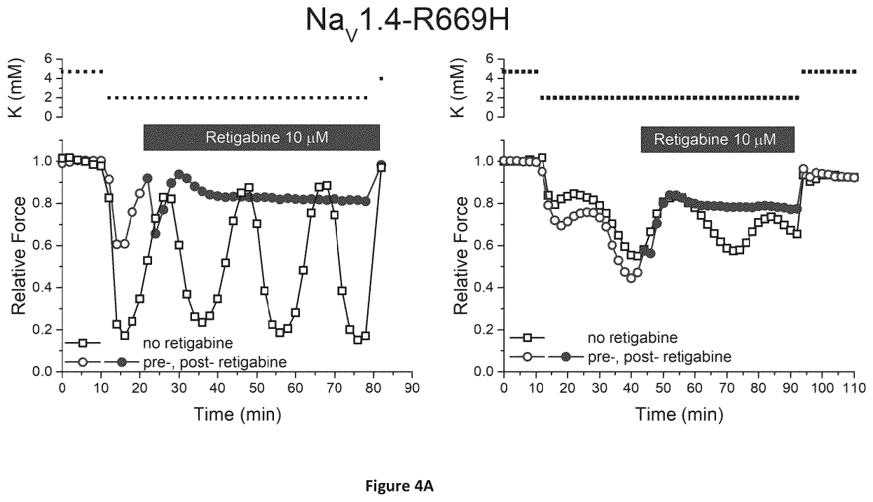

The dose-response relation for the relative protection from loss of force by pre-treatment with retigabine is shown in FIG. 3. The concentration-dependent protection had an equivalent K_{d}=0.82±0.13 mM and 2.2±0.42 mM for Naᵥ1.4-R669H and Caᵥ1.1-R528H, respectively, based on the best-fit by a single binding-site model. A limited series of measurements were also performed with pinacidil, a K_{ATP} channel opener, that has previously been shown to protect against loss of force for an ex vivo contraction assay of human HypoPP muscle at a concentration of 100 mM. Using the Naᵥ1.4-R669H mouse model, it is confirmed that pre-treatment with pinacidil protects HypoPP muscle from a loss of force in a low-K⁺ challenge (FIG. 3), however the potency was about 10-fold lower (K_{d}=8.1±0.5 mM) compared to retigabine.

It was also tested whether application of retigabine would hasten the recovery from an on-going low-K⁺ induced loss of force in HypoPP muscle. The paired response from the contralateral muscle, without drug exposure, was used as an internal control. FIG. 4A shows two examples from soleus muscles of separate Naᵥ1.4-R669H HypoPP mice. A 2 mM K⁺ challenge was applied first, and then after confirming a loss of peak force, the bath was exchanged by a new 2 K⁺ solution that also contained 10 mM retigabine. Application of retigabine at 10 min. after the onset of reduced force (FIG. 4A, left panel) or even after 30 min when two loss-of-force cycles were complete (FIG. 4A, right panel), produced a sustained improvement in force and prevented any further cyclical reduction of force. The improved level of tetanic force in retigabine, while still in a 2 mM K⁺ bath, was on average 83.2%±0.021 (n=3) of baseline, which is comparable to the performance of WT soleus in 2 mM K⁺. Rescue by retigabine from an on-going loss of force in low K⁺ was also observed for the EDL muscle of Caᵥ1.1-R5228H mice (FIG. 4 B).

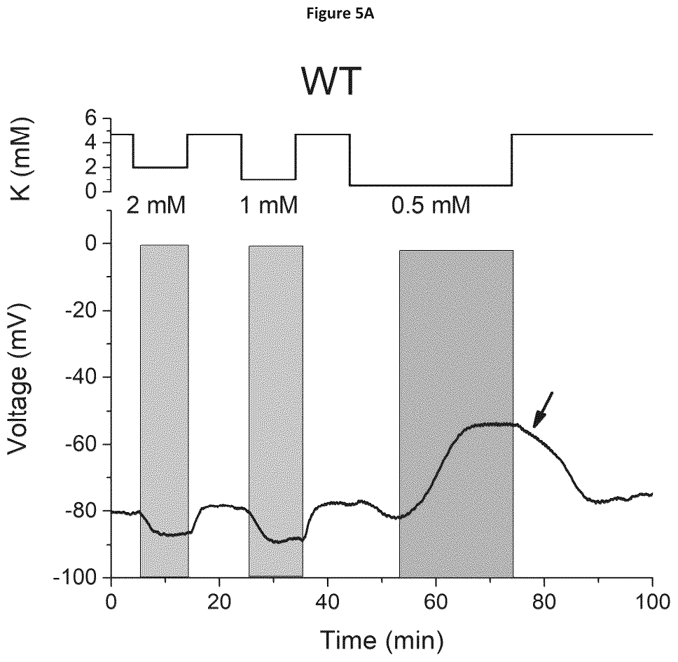

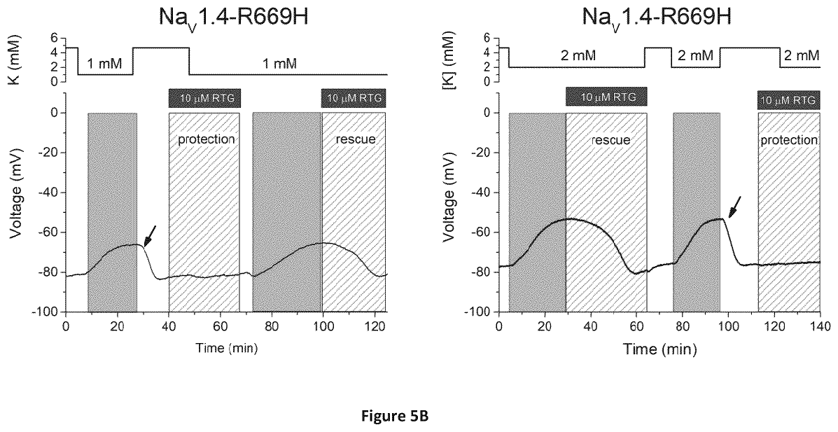

The impairment of contraction during an episode of HypoPP is caused by sustained depolarization, which inactivates sodium channels and reduces muscle fiber excitability. In HypoPP, ictal depolarization of the resting potential (Vᵣₑₛₜ) paradoxically occurs in low K⁺ that would normally hyperpolarize the membrane from a negative shift of the K⁺ Nernst potential (E_{K}). It was tested whether retigabine prevents or reverses this paradoxical depolarization of HypoPP fibers exposed to low K⁺. FIG. 5 shows the K⁺-induced changes of Vᵣₑₛₜ for EDL fibers impaled with a sharp microelectrode. Reduction of K⁺ to 2 mM or even 1 mM caused hyperpolarization of WT fibers, as expected from the negative shift of E_{K} (FIG. 5A, gray shaded regions). At an extremely low K⁺ of 0.5 mM, even WT fibers paradoxically depolarized (magenta region) as previously described. For EDL fibers from the Naᵥ1.4-R669H mouse, the more modest K⁺ reductions to 2 mM or 1 mM elicited paradoxical depolarization, which is the membrane potential equivalent of ictal HypoPP. Paradoxical depolarization of WT or of HypoPP fibers was reversible upon return to 4.7 mM K⁺ (FIG. 5 arrows), which shows this behavior was not caused by fiber damage. Addition of retigabine before the low K⁺ challenge prevented the paradoxical depolarization of HypoPP fibers (FIG. 5B, hatched regions labeled protection). Moreover, HypoPP fibers that were paradoxically depolarized in low K⁺ repolarized to the normal Vᵣₑₛₜ when retigabine was added to the bath, even though the low K⁺ condition was unchanged (FIG. 5B, hatched regions labeled rescue). These observations support the view that the beneficial effects of retigabine occur by a stabilizing effect on the normally polarized value of Vᵣₑₛₜ.

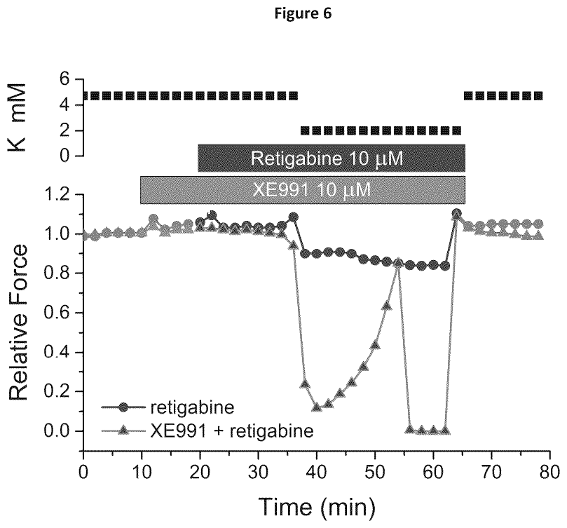

To confirm the beneficial effects of retigabine were dependent upon currents conducted by Kᵥ7-type K⁺ channels, the effect of a Kᵥ7 inhibitor, XE991, was assessed. As shown in FIG. 6, exposure to 10 mM XE991 did not alter the peak tetanic force in 4.7 mM K⁺ (10 to 20 min, magenta symbols). The other soleus muscle from the same HypoPP mouse was not exposed to XE991. Retigabine was then applied to both soleus muscle preparations, followed by a low-K⁺ challenge. A marked loss of force in low K⁺ occurred for the XE991+ retigabine exposed soleus, whereas the paired HypoPP muscle was protected by retigabine. These data show the beneficial effect of retigabine is blocked by XE991 and suggest that basal activity of Kᵥ7 channels limits the severity of the loss of force in low K⁺.

As shown in herein, the therapeutic potential of K⁺ channel openers to reduce the frequency and severity for the attacks of weakness in periodic paralysis has been recognized for over 30 years. The fundamental principle is that because increasing the membrane conductance to K⁺ will hyperpolarize the membrane potential toward E_{K}≈−95 mV, this class of drugs will counteract the sustained anomalous depolarization of the resting potential that causes transient weakness in periodic paralysis by inactivation of sodium channels. Many criteria must be met, however, for this strategy to be safe and effective. For example, many K⁺ channels are activated by depolarization, and the drug-induced augmentation of channel opening must occur over an operational voltage range that is relevant for stabilization of a normal resting potential (−80 to −95 mV) and extend to the anomalous depolarization during paralysis (typically −50 to −60 mV). This voltage-dependent property will determine whether the increased K⁺ conductance will primarily affect Vᵣₑₛₜ, the action potential waveform (e.g. amplitude and duration), or both. Another concern is the expression pattern across different tissues for the K⁺ channels that are activated by the drug; expression in skeletal muscle for the desired effect, with lower expression in other tissues to minimize side effects. The drug potency (small dissociation constant, K_{d}) and magnitude of the K⁺ conductance increase are critical as well. If the conductance increase is very small, then the drug may not produce a meaningful reduction in the susceptibility to attacks of weakness. Conversely, a very large K⁺ conductance increase may impair contractility by reducing muscle fiber excitability.

The effectiveness of retigabine at low mM concentrations to protect HypoPP muscle from a loss of force in a low-K⁺ challenge (2 mM) demonstrates the therapeutic potential of Kᵥ7.x channel openers in the symptomatic management of HypoPP. The Kᵥ7.1-Kᵥ7.5 family of voltage-gated K⁺ channels is encoded by KCNQ1-KCNQ5 genes, so-named because the founding member, KCNQ1, was identified as a disease locus for type 1 long QT syndrome (LQT1). The predominant Kᵥ7 channel in the heart is Kᵥ7.1, and importantly, this isoform is 100-fold less sensitive to retigabine than Kᵥ7.2-Kᵥ7.5, which explains the absence of cardiac side effects. The Kᵥ7.2-Kᵥ7.5 isoforms were initially characterized as “neuronal”, with Kᵥ7.2, Kᵥ7.3, Kᵥ7.5 expressed in the central nervous system, Kᵥ7.4 in the cochlea, and Kᵥ7.2 in the peripheral nervous system. Skeletal muscle was not initially considered to be a site of significant Kᵥ7.x expression, but subsequent studies using mouse and human skeletal muscle have detected Kᵥ7.1-Kᵥ7.5 transcripts by RT-PCR, and Kᵥ7.2-Kᵥ7.4 subunit protein by both immunoblot and immunohistochemistry. Changes in the expression pattern of Kᵥ7.x subunits with muscle proliferation and differentiation have implicated a role in development.

A physiological role has not yet been established for the voltage-gated K⁺ current conducted by Kᵥ7 channels in skeletal muscle. Voltage-clamp studies to characterize Kᵥ7.x currents in muscle have not been reported, most likely because of the technical difficulty in isolating this component of the K⁺ current from the contributions by a multitude of K⁺ channels in this tissue (over 20 described). The beneficial effect of retigabine is completely prevented by XE991 (FIG. 6), which at 10 mM is a specific inhibitor of Kᵥ7 channels and supports the interpretation that the K⁺ channel-opening action of retigabine is the mechanism of drug action. Because low-dose retigabine protects HypoPP muscle from a low-K⁺ induced loss of force and also suppresses after-discharges in mouse models of myotonia (using pharmacologic block of the chloride conductance or by disruption of the Clcn1 gene), the Kᵥ7.x K⁺ current clearly has an important role in regulating muscle excitability. Prior experiments (using current-clamp) to explore the mechanism for retigabine-induced suppression of myotonia did not detect a change in fiber input resistance, resting potential or action potential properties of mouse muscle upon exposure to 20 mM retigabine. The present data corroborates the observation that retigabine does not affect Vᵣₑₛₜ of normally polarized muscle fibers (FIG. 5B, hatched regions of protection where Vᵣₑₛₜ in 4.7 K⁺ did not change when retigabine was added). Most likely, the absence of an observable drug-induced shift of Vᵣₑₛₜ is because in normally polarized fibers this resting voltage is nearly equal to E_{K}. Electrophysiological approaches with greater sensitivity and specificity will be required to elucidate the normal role of Kᵥ7.x K⁺ currents in muscle and the beneficial effect of activation by retigabine.

While the chronic use of retigabine as a prophylactic antiepileptic drug for refractory partial-onset seizures was discontinued in the US in 2017 because of adverse effects (primarily skin discoloration and also CNS effects with dose-dependent dizziness, somnolence, headache), the enthusiasm for Kᵥ7 channel activators remains strong for management of epilepsy (especially KCNQ2 developmental and epileptic encephalopathies), pain management, for neuroprotection in degenerative diseases, and for mood stabilization. Derivatives of retigabine with reduced dimer formation and therefore lack of skin discoloration are in clinical trials for KCNQ2-DDE (XEN496 in children; NCT04912856) and for focal epilepsy in adults (XEN1101; NCT03796962). Another Kᵥ7 opener, GRT-X, is chemically unrelated to retigabine and is in development. These pipeline drugs also act by activation of Kᵥ7 channels, and therefore are likely to be effective in ameliorating attacks of periodic paralysis.

Moreover, the present data showing rescue of both contractility (FIG. 4) and of Vᵣₑₛₜ (FIG. 5B) demonstrate the potential for short-term administration of retigabine, or newer Kᵥ7 openers, as abortive therapy for an acute attack of weakness in HypoPP. After a single 100 mg oral dose, a Cₘₐₓ of 390 ng/ml occurs within 1.5 hrs., and the half-life is 8 hrs. An unbound retigabine fraction of 0.9 mM will be achieved with a single 400 mg dose (assumes 80% protein binding). Based on this observation, a clinical trial for efficacy of Kᵥ7 openers could be performed by assessing the rate of recovery of the compound muscle action potential in the long exercise test for periodic paralysis, as was done to studies of bumetanide. The functional defect in periodic paralysis operates like a binary switch, with a sustained shift between the normal Vᵣₑₛₜ and an anomalously depolarized value. Consequently, a single dose of retigabine may be sufficient to “reset” Vᵣₑₛₜ to the normal range and suppress a recurrent attack of weakness over the minutes to hours required for correction of extracellular [K⁺].

An adverse effect of retigabine on skeletal muscle function is unlikely. One report described a dose-dependent reduction of contractile force during a 3 sec tetanic stimulation of rat muscle, with an IC₅₀ of 1 mM, presumably from a reduced fiber excitability. Studies with up to 20 mM retigabine show no impairment of peak isometric force during a 400 msec tetanic contraction, applied once every two minutes for two hrs. Similarly, the prior two studies on the effect of retigabine in mouse models of myotonia did not report a reduction of ex vivo tetanic force with 20 or 30 mM retigabine. In vivo mouse and rat studies that used a very high dose of retigabine (30 mg/kg which is 5.7 times larger than the maximal oral dose of 400 mg for a 70 kg human) showed a loss of force with tetanic contractions lasting seconds, but this result is not likely to be relevant to patient care. It is believed that the “fatigue” described as an adverse effect of retigabine in AED trials is more likely to be of central origin rather than muscle fiber dysfunction.

### Example 2

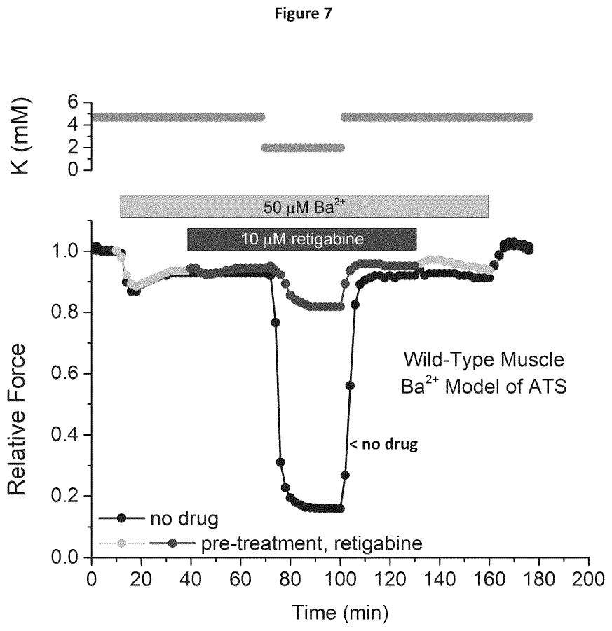

A pharmacological model of Andersen-Tawil Syndrome (ATS) is created, in vitro, with a low concentration of barium (50 μM), which blocks Kir potassium channels. Kir2.1 is deficient from loss of function mutations of the KCNJ2 gene in ATS. FIG. 7 shows that a low K challenge (2 mM) produces a profound loss of force in the ATS model, whereas pre-administration of 10 μM retigabine is effective a preventing the loss of muscle force that occurs with a low K challenge.

### Example 3

Based on the observation disclosed herein, a clinical trial for efficacy of KV7 channel openers can be performed by assessing the rate of recovery of muscle action potential in the long exercise test for periodic paralysis, as was use in another study (Jitpimolmard, N., E. Matthews, and D. Fialho. (2020). Treatment Updates for Neuromuscular Channelopathies. Curr Treat Options Neurol. 22(10): p. 34).

Enrolment criteria include, for inclusion, at least 18 years of age; diagnosis of genetically confirmed HypoPP; clinical symptoms or signs of active symptomatic disease (at least 1 attack in last 12 months); and practicing an acceptable method of birth control for the duration of the trial. Exclusion criteria include inability or unwillingness to provide informed consent; older than 64 years old; other conditions causing hand weakness which could interfere with study measurements (e.g. due to a stroke, trauma or arthritis); patients with a history of cardiac disease, renal failure or moderate to severe hepatic disease (recognizing abnormalities in serum transaminases are common in people with HypoPP as they arise from skeletal muscle rather than any specific liver abnormality; consequently, raised serum bilirubin>20% above the baseline value will be used to identify abnormal liver function); women who are pregnant or breast-feeding; patients with a current or previous history of diabetes, porphyria, symptomatic hypotension; patients known to be allergic retigabine or its excipients; patients with a history of inadequately treated Addison's disease; patients participating in another interventional trial in the previous 1 month.

Such protocol involves two assessment visits at approximately four weeks apart. Following baseline assessments, a localized attack of weakness will be induced by isometric exercise of the abductor digit minimi (ADM) in the hand as per the McManis Long-Exercise Test EMG protocol (McManis, P. G., Lambert, E. H. and Daube, J. R. (1986) ‘The exercise test in periodic paralysis’: Muscle Nerve, 9, 704-10). Participants will be randomly assigned to either Kᵥ7 channel opener or placebo for the first visit. Identical appearing capsules will be prepared to blind both researcher and participant to treatment allocation. The assigned treatment will be taken by mouth at the onset of a focal attack defined as 40% decrement in ADM CMAP amplitude compared to the maximum CMAP amplitude recorded during or after the exercise. During admission, each patient will be monitored according to the research protocol. At the end of the assessment protocol the participant will be discharged home. The duration of each admission will be approximately 6 hours. The second assessment will follow an identical protocol to the first, but with the other treatment administered.

Primary Outcome Measures: Focal attack severity one hour after treatment [Time Frame: one hour after treatment]. This will be measured as CMAP amplitude expressed as a percent of peak CMAP during or after the McManis exercise.

Secondary Outcome Measures: 1) Focal attack duration (Time Frame: 4 hours). This will be measured as the time between treatment administration until CMAP returns to 65% of peak CMAP within 4 hours following the treatment intake. 2) The initial effect of treatment on severity of a focal attack (Time Frame: The initial effect of treatment on severity of a focal attack within the first two hours post treatment). The effect of treatment on severity of a focal attack within the first two hours (0-2). This will be measured as CMAP amplitude (in percent compared to peak) area under the curve (AUC) from treatment administration until two hours post-treatment. 3) The late effect of treatment on severity of a focal attack (Time Frame: The late effect of treatment on severity of a focal attack two to four hours post-treatment). The effect of treatment on severity of a focal attack within the last 2 hours (3-4). This will be measured as CMAP amplitude (in percent) AUC from treatment administration during the third and the fourth hours post-treatment. 4) Safety of Kᵥ7 channel opener, assessed by vital signs, physical exam, potassium levels and self-reported adverse events (Time Frame: Safety of Kᵥ7 channel opener in HypoPP within 7 days of each study visit). Baseline instantaneous potassium measurements as well as laboratory measurements, vital signs (blood pressure/heart rate) and a physical exam including MRC score are done prior to exercise and IMP intake. During the first 4 hours following IMP intake vital signs (blood pressure/heart rate) and instantaneous serum potassium levels are measured frequently as per protocol. Any reported symptoms or adverse events are recorded. In addition, intermittent electrophysiological recordings are taken from the non-exercised hand in order to identify the development of a major attack of paralysis early. At the end of the observation period (4 hours after Kᵥ7 channel opener intake) serum potassium levels are measured by the local hospital laboratory and a physical exam is performed including an MRC score. Safety is also assessed by phone call evaluating adverse events reported by the participants and recorded in a diary occurring within 1 week following each visit.

Retigabine is shown to reduce severity and accelerate recovery from episodes of muscle weakness in patients with hypokalemic periodic paralysis.

### Example 4

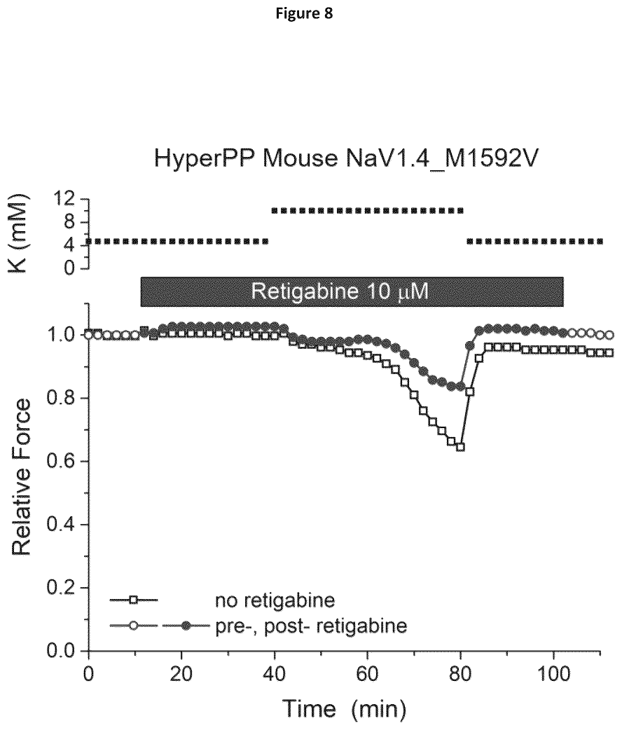

Retigabine is partially effective in preventing the loss of force in our mouse model of hyperkalemic periodic paralysis (HyperPP). FIG. 8 shows an in vitro contraction test for the soleus muscle obtained from the knock-in mutant mouse model of HyperPP (Wu et al. J Clin Invest 2012 December; 122(12):4580-91). The two paired muscles (left and right) were tested in separate organ baths. Retigabine (10 μM) was applied to one bath beginning at 10 min (filled circles) and the maximal force remained comparable to control (i.e. about 1.0). At 40 min into the study, a high K challenge was performed by exchanging the 4.7 mM K+ bath with one containing 10 mM K+. The control HyperPP soleus muscle had a 40% loss of force (open circles), whereas pre-treatment with retigabine attenuated the loss of force by about 50%. Return of the bath K+ to 4.7 mM (80 min in the figure) elicits a full recovery of force for both the control and retigabine exposed muscles.

### Example 5

Another KV7 channel opener, XEN1101, provides complete protection from the loss of force in the NaV1.4-R669H mouse model of HypoPP. FIG. 9 shows the response for the in vitro contraction assay for paired soleus muscles (left and right) harvested from the same HypoPP NaV1.4-R669H mouse. Addition of XEN1101 (10 μM at 10 min time point) produced a 20% increase in baseline force, most likely because a spontaneous partial attack of weakness was already underway in both muscles. Provocation with a low K channels (2 mM at 40 min) induced a profound oscillatory loss of force for control HypoPP muscle whereas no decrement of force occurred for the XEN1101 exposed soleus muscle.

1. A method of preventing or treating a skeletal muscle disease in a subject comprising administering to the subject an effective amount of a potassium channel opener.

2. The method of claim 1 wherein the skeletal muscle disease is an inherited skeletal muscle disease or a sporadic skeletal muscle disease.

3. The method of claim 2 wherein the inherited skeletal muscle disease is periodic paralysis.

4. The method of claim 3 wherein the periodic paralysis is hypokalemic periodic paralysis, hyperkalemic periodic paralysis, Andersen Tawil syndrome (ATS), or thyrotoxic periodic paralysis.

5. The method of claim 4 wherein the thyrotoxic periodic paralysis is endogenous or caused by exogenous thyroid hormone administration.

6. The method of claim 1 wherein the potassium channel opener is a Kᵥ7 channel opener.

7. The method of claim 6 wherein the Kᵥ7 channel opener is a KCNQ2/3 (Kᵥ7.2/7.3), KCNQ2, KCNQ3, KCNQ4 or KCNQ5 potassium channel opener or any combination thereof.

8. The method of claim 6 wherein the Kᵥ7 channel opener is selected from retigabine, (10,10-bis(4-pyridinylmethyl)-9(10H)-anthracenone dihydrochloride), N-[4-(6-fluoro-3,4-dihydro-1H-isoquinolin-2-yl)-2,6-dimethylphenyl]-3,3-dimethylbutanamide, (N-[(3-fluorophenyl)-methyl]-1-(2-methoxyethyl)-4-methyl-2-oxo-(7-trifluoromethyl)-1H-quinoline-3-carboxylic acid amide), N-[4-(6-fluoro-3,4-dihydro-1H-isoquinolin-2-yl)-2,6-dimethylphenyl]-3,3-dimethylbutanamide, or flupirtine, or an analogue of any of the foregoing.

9. The method of claim 1 wherein the effective amount comprises administering the potassium channel opener from between about 30 minutes prior to consuming food until 12 hours after consuming food.

10. The method of claim 1 wherein the preventing or treating is abortive therapy or prophylactic therapy.

11. The method of claim 10 wherein the abortive therapy is achieved with a single dose of potassium channel opener.

12.-14. (canceled)

15. The method of claim 1 further comprising administering to the subject a potassium-sparing diuretic, a potassium supplement, a carbonic anhydrase inhibitor, a sodium channel blocker, or any combination thereof.

16.-20. (canceled)

21. A therapeutic combination comprising a potassium channel opener and a carbonic anhydrase inhibitor.

22.-24. (canceled)

25. The therapeutic combination of claim 21 further comprising a potassium supplement, a potassium sparing diuretic, a sodium channel blocker, or any combination thereof.

26.-28. (canceled)

29. A therapeutic combination comprising a potassium channel opener and a potassium supplement.

30.-31. (canceled)

32. The therapeutic combination of claim 29 further comprising a carbonic anhydrase inhibitor, a potassium sparing diuretic, a sodium channel blocker, or any combination thereof.

33.-35. (canceled)

36. A therapeutic combination comprising a potassium channel opener and a potassium sparing diuretic.

37.-38. (canceled)

39. The therapeutic combination of claim 36 further comprising a carbonic anhydrase inhibitor, a potassium supplement, a sodium channel blocker, or any combination thereof.

40.-42. (canceled)

43. A therapeutic combination comprising a potassium channel opener and a sodium channel blocker.

44.-45. (canceled)

46. The therapeutic combination of claim 43 further comprising a carbonic anhydrase inhibitor, a potassium supplement, a sodium channel blocker, or any combination thereof.

47.-49. (canceled)

50. A therapeutic combination comprising a potassium channel opener, a carbonic anhydrase inhibitor, a potassium supplement and a potassium-sparing diuretic.

51. A method of preventing or treating a skeletal muscle disease in a subject comprising administering to the subject an effective amount of a therapeutic combination of a potassium channel opener and a carbonic anhydrase inhibitor.

52.-66. (canceled)

67. The method of claim 51 further comprising further administering to the subject a potassium-sparing diuretic, a potassium supplement, a sodium channel blocker, or any combination thereof.

68.-73. (canceled)
